(19) United States
(12) Patent Application Publication
(10) Pub. No.: US 2024/0159761 A1
(43) Pub. Date: May 16, 2024

(54) BIOMARKERS FOR IDENTIFYING AND TREATING CANCER PATIENTS

(71) Applicant: Mayo Foundation for Medical Education and Research, Rochester, MN (US)

(72) Inventors: Svetlana Bornschlegl, Rochester, MN (US); Allan B. Dietz, Chatfield, MN (US); Michael P. Gustafson, Scottsdale, AZ (US)

(21) Appl. No.: 18/280,863

(22) PCT Filed: Mar. 9, 2022
(86) PCT No.: PCT/US2022/019487

§ 371 Date: Sep. 7, 2023

Related U.S. Application Data
(60) Provisional application No. 63/158,740, filed on Mar. 9, 2021.

(51) Int. Cl. G01N 33/574; A61K 39/00; A61P 35/00; C07K 16/28

(52) U.S. Cl. CPC G01N 33/57484; A61P 35/00; C07K 16/2818; A61K 2039/505

(57) ABSTRACT

Methods and materials for identifying mammals having cancer as being likely to respond to treatment with a checkpoint inhibitor are provided herein. For example, materials and methods for using immune profiling with particular biomarkers on circulating patient immune cells to predict patient response to checkpoint inhibitors (e.g., pembrolizumab) are provided herein.

### CROSS-REFERENCE TO RELATED APPLICATIONS

This application claims benefit of priority from U.S. Provisional Application No. 63/158,740, filed on Mar. 9, 2021. The disclosure of the prior application is considered part of (and is incorporated by reference in) the disclosure of this application.

### TECHNICAL FIELD

This document relates to methods and materials for identifying mammals having cancer as being more likely to respond to treatment with a checkpoint inhibitor. For example, this document relates to materials and methods for using immune profiling with particular biomarkers on circulating patient immune cells to predict patient response to the checkpoint inhibitor, pembrolizumab.

### BACKGROUND

Inhibitors to the checkpoint proteins cytotoxic T-lymphocyte-associated protein 4 (CTLA-4) and programmed death 1 (PD-1) are used in cancer treatment. However, a lack of understanding of the patient response to treatment limits accurate identification of potential responders to immunotherapy.

### SUMMARY

This document provides methods and materials for determining the usefulness of particular drugs by matching the likelihood that a drug will be effective with a mammal's particular immune system status. For example, certain drugs can manipulate immunity to drive toward an anti-tumor immune response. However, the immune system varies between individuals with cancer, and that variability can be exacerbated by the underlying tumor(s). The methods provided herein can reconcile and identify the immune system variability, and can identify mammals in which the drugs are likely to be effective.

As disclosed herein, expression of PD-1 and CTLA-4 on 19 leukocyte populations was measured in the peripheral blood of cancer patients, and also in healthy volunteers to determine the normal expression patterns for these checkpoint proteins. Unsupervised hierarchical clustering found four immune profiles shared across solid tumor types, while chronic lymphocytic leukemia patients had a largely unique immune profile. Expression of PD-1 and CTLA-4 on the leukocyte populations also was measured on a cohort of cancer patients receiving a PD-1 inhibitor (pembrolizumab) in order to identify differences between responders, non-responders, and healthy controls. As demonstrated herein, cancer patients had pre-treatment PD-1 and CTLA-4 expression on their leukocyte populations at different levels than healthy volunteers, and two leukocyte populations positive for CTLA-4 were identified that had not been previously described. In addition, higher levels of PD-1⁺CD3⁺CD4⁻CD8⁻ cells were observed in patients with progressive disease, identifying these cells as a potential biomarker of response. The results presented herein suggest that categorization of patients based on immune profiles can differentiate responders from non-responders to immunotherapy for solid tumors.

Having the ability to identify patients based on their immune systems (and independent of their underlying pathology) provides a unique opportunity to determine the best course of therapy for patients who are undergoing immunotherapy in general and PD-1 based therapy in particular. The methods provided herein are not invasive, but rather use peripheral blood processed with a tool such as multi-parameter flow cytometry analysis to categorize patients. The methods also can provide a means for more advanced categorization of patients and for determining a dose sufficient to manifest the appropriate response, and also for choosing therapeutics that will be more successful in treating disease by providing a targeted approach that directly compliments the patient's immune system.

In general, one aspect of this document features methods for identifying a mammal having cancer as being likely to respond to treatment with pembrolizumab. The methods can include, or consist essentially of, determining that a biological sample from the mammal has an elevated level of an immune phenotype associated with a likely response to treatment with pembrolizumab, as compared to a control level of the immune phenotype, and identifying the mammal as being likely to respond to treatment with pembrolizumab. The mammal can be a human. The immune phenotype can be selected from the group consisting of PD1+CD8+ Naïve (% CD8), number of granulocytes (Grans) (cells/μL), number of Neutrophils 15+16+(cells/μL), Grans (% CD45), Eosinophils 15+16− (cells/μL), PD1+CD3+ (% MNCs), and PD1+CD8+ (% CD3). The cancer can be selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

In another aspect, this document features methods for assessing a mammal having cancer. The methods can include, or consist essentially of, (a) detecting the presence of an elevated level of an immune phenotype in a biological sample from the mammal, as compared to a control level of the immune phenotype, wherein the immune phenotype is associated with a likely response to treatment with pembrolizumab, and (b) classifying the mammal as being likely to respond to treatment with pembrolizumab. The mammal can be a human. The immune phenotype can be selected from the group consisting of PD1+CD8+ Naïve (% CD8), number of granulocytes (Grans) (cells/μL), number of Neutrophils 15+16+ (cells/μL), Grans (% CD45), Eosinophils 15+16− (cells/μL), PD1+CD3+ (% MNCs), and PD1⁺CD8+ (% CD3). The cancer can be selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

In another aspect, this document features methods for identifying a mammal having cancer as not being likely to respond to treatment with pembrolizumab. The methods can include, or consist essentially of, determining that a biological sample from the mammal has an elevated level of an immune phenotype associated with a lack of response to treatment with pembrolizumab, as compared to a control level of the immune phenotype, and identifying the mammal not as being likely to respond to treatment with pembrolizumab. The mammal can be a human. The immune phenotype can be selected from the group consisting of PD1+CD3+ DN (% CD3), Intermediate Monocytes 14+16+ (cells/μL), CD3 (% MNCs), and Monocytes (% CD45pos). The cancer can be selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

In another aspect, this document features methods for assessing a mammal having cancer. The methods can include, or consist essentially of, (a) detecting the presence of an elevated level of an immune phenotype in a biological sample from the mammal, as compared to a control level of the immune phenotype, wherein the immune phenotype is associated with a lack of response to treatment with pembrolizumab, and (b) classifying the mammal as not being likely to respond to treatment with pembrolizumab. The mammal can be a human. The immune phenotype can be selected from the group consisting of PD1⁺CD3+ DN (% CD3), Intermediate Monocytes 14+16+ (cells/μL), CD3 (% MNCs), and Monocytes (% CD45pos). The cancer can be selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

In another aspect, this document features methods for assessing a mammal identified as having cancer. The methods can include, or consist essentially of, (a) performing flow cytometry to detect the presence of an elevated level of an immune phenotype associated with a likely response to treatment with pembrolizumab, as compared to a control level of the immune phenotype, and (b) classifying the mammal as being likely to respond to treatment with pembrolizumab based at least in part on the detected presence of the immune phenotype. The mammal can be a human. The immune phenotype can be selected from the group consisting of PD1+CD8+ Naïve (% CD8), number of granulocytes (Grans) (cells/μL), number of Neutrophils 15+16+(cells/μL), Grans (% CD45), Eosinophils 15+16− (cells/μL), PD1+CD3+ (% MNCs), and PD1+CD8+ (% CD3). The cancer can be selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

In another aspect, this document features methods for assessing a mammal identified as having cancer. The methods can include, or consist essentially of, (a) performing flow cytometry to detect an elevated level of an immune phenotype associated with a lack of response to treatment with pembrolizumab, as compared to a control level of the immune phenotype, and (b) classifying the mammal as not being likely to respond to treatment with pembrolizumab based at least in part on the elevated level of the immune phenotype. The mammal can be a human. The phenotype can be selected from the group consisting of PD1+CD3+ DN (% CD3), Intermediate Monocytes 14+16+ (cells/μL), CD3 (% MNCs), and Monocytes (% CD45pos). The cancer can be selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

In another aspect, this document features methods for treating a mammal having cancer. The methods can include, or consist essentially of, (a) identifying the mammal as having an elevated level of an immune phenotype associated with a likely response to treatment with pembrolizumab, as compared to a control level of the immune phenotype, and (b) administering, to the mammal, or instructing the mammal to self-administer, a composition containing pembrolizumab. The mammal can be a human. The immune phenotype can be selected from the group consisting of PD1+CD8+ Naïve (% CD8), number of granulocytes (Grans) (cells/μL), number of Neutrophils 15+16+ (cells/μL), Grans (% CD45), Eosinophils 15+16− (cells/μL), PD1+CD3+ (% MNCs), and PD1+CD8+ (% CD3). The cancer can be selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

In another aspect, this document features methods for treating a mammal having cancer. The methods can include, or consist essentially of, administering a composition containing pembrolizumab to a mammal identified as having the an elevated level of an immune phenotype associated with a likely response to pembrolizumab treatment, as compared to a control level of the immune phenotype. The mammal can be a human. The immune phenotype can be selected from the group consisting of PD1+CD8+ Naïve (% CD8), number of granulocytes (Grans) (cells/μL), number of Neutrophils 15+16+ (cells/μL), Grans (% CD45), Eosinophils 15+16− (cells/μL), PD1⁺CD3+ (% MNCs), and PD1⁺CD8+ (% CD3). The cancer can be selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

In another aspect, this document features methods for treating a mammal having cancer. The methods can include, or consist essentially of, (a) identifying the mammal as having an elevated level of an immune phenotype associated with a lack of response to treatment with pembrolizumab, as compared to a control level of the immune phenotype, and (b) administering, to the mammal, or instructing the mammal to self-administer, a composition containing a therapeutic agent other than pembrolizumab. The mammal can be a human. The immune phenotype can be selected from the group consisting of PD1+CD3+ DN (% CD3), Intermediate Monocytes 14+16+ (cells/μL), CD3 (% MNCs), and Monocytes (% CD45pos). The cancer can be selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

In still another aspect, this document features methods for treating a mammal having cancer. The methods can include, or consist essentially of, administering a composition containing a therapeutic agent other than pembrolizumab to a mammal identified as having an elevated level of an immune phenotype associated with a lack of response to pembrolizumab treatment, as compared to a control level of the immune phenotype. The mammal can be a human. The immune phenotype can be selected from the group consisting of PD1+CD3+ DN (% CD3), Intermediate Monocytes 14+16+ (cells/μL), CD3 (% MNCs), and Monocytes (% CD45pos). The cancer can be selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

This document also features methods for determining a Single Analyte Immune Data (SAID) score for a mammal with cancer. The methods can include, or consist essentially of, measuring, in a biological sample from the mammal, the following immune phenotypes:
PD1+CD3+ DN (% CD3);
PD1+CD8+ Naïve (% CD8);
Granulocytes (Grans) (cells/μL);
Neutrophils 15+16+ (cells/μL);
Grans (% CD45);
Eosinophils 15+16− (cells/μL);
PD1+CD3+ (% MNC);
PD1+CD8+ (% CD3);
Intermediate Monocytes 14+16+ (cells/μL);
CD3 (% MNC); and
Monocytes (% CD45pos),
assigning a first score of −2.5 when the PD1+CD3+ DN (% CD3) is 19 or above, or assigning a first score of 0 when the PD1+CD3+ DN (% CD3) is less than 19;
assigning a second score of +1 when the PD1+CD8+ Naïve (% CD8) is 10 or above, or assigning a second score of 0 when the PD1+CD8+ Naïve (% CD8) is less than 10;
assigning a third score of +1 when the Grans (cells/μL) is 4500 cells/μL or above, or assigning a third score of 0 when the Grans (cells/μL) is less than 4500;
assigning a fourth score of +1 when the Neutrophils 15+16+ (cells/μL) is 4300 or above, or assigning a fourth score of 0 when the Neutrophils 15+16+ (cells/μL) is less than 4300;
assigning a fifth score of +1 when the Grans (% CD45) is 78 or above, or assigning a fifth score of 0 when the Grans (% CD45) is less than 78;
assigning a sixth score of +0.5 when the Eosinophils 15+16− (cells/μL) is 271 or above, or assigning a sixth score of 0 when the Eosinophils 15+16− (cells/μL) is less than 271;
assigning a seventh score of +0.5 when the PD1+CD3+ (% MNC) is 13 or above, or assigning a seventh score of 0 when the PD1+CD3+ (% MNC) is less than 13;
assigning an eight score of +0.5 when the PD1+CD8+ (% CD3) is 26 or above, or assigning an eighth score of 0 when the PD1+CD8+ (% CD3) is less than 26;
assigning a ninth score of −1 when the Intermediate Monocytes 14+16+(cells/μL) is 88 or above, or assigning a ninth score of 0 when the Intermediate Monocytes 14+16+ (cells/μL) is less than 88;
assigning a tenth score of −0.5 when the CD3 (% MNC) is 52 or above, or assigning a tenth score of 0 when the CD3 (% MNC) is less than 52;
assigning an eleventh score of −0.5 when the Monocytes (% CD45pos) is 35 or above, or assigning an eleventh score of 0 when the Monocytes (% CD45pos) is less than 35; and
calculating the SAID score by totaling the first score through the eleventh score.

In another aspect, this document features methods for identifying a mammal having cancer as being more likely to respond to treatment with pembrolizumab or as being less likely to respond to treatment with pembrolizumab. The methods can include, or consist essentially of, determining a SAID score for the mammal as described herein, and identifying the mammal as being more likely to respond to the treatment with pembrolizumab when the SAID score is 1 or greater, or identifying the mammal as being less likely to respond to the treatment with pembrolizumab when the SAID score is less than 1.

In another aspect, this document features methods for treating a mammal having cancer. The methods can include, or consist essentially of, using the methods described herein to calculate a SAID score for the mammal, wherein the SAID score is 1 or greater, and administering pembrolizumab to the mammal.

In another aspect, this document features methods for treating a mammal having cancer. The methods can include, or consist essentially of, using the methods described herein to calculate a SAID score for the mammal, wherein the SAID score is less than 1, and administering a treatment other than pembrolizumab to the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C: Characterization of T-Cell Signaling Markers, CD152 PE, and PD-1 PC7 Antibody Validation. FIG. 1A: A whole blood sample from a healthy volunteer was stained with the T-cell signaling panel. Histograms were generated from each of 10 antibodies (except CD45) and used to delineate mononuclear populations (defined by CD45⁺ side scatter (SSC) lo/med). In most cases, two regions (R1 and R2) were created for each peak of expression, including peaks with no expression (i.e., N). Forward scatter (FS) and side scatter density plots were created for each histogram peak. Together, the specific peaks were used to identify unique phenotype combinations. FIG. 1B: Validation of CD152 was done with isolated PBMCs. A fraction of the cells were stained directly after isolation (Day 0). The remaining cells were cultured for 24 hours (Day 1) with and without CD3CD28 DynaBeads. Stimulated cells show increased CD152 levels. Histograms were generated to show stimulation-induced CD152, as seen by R1, which was not seen among healthy volunteers. The three samples were overlaid. FIG. 1C: PD-1 antibody was validated by blocking PBMCs with anti-PD-1 antibody before the addition of T-cell signaling mix. An FMO for PD-1 was performed on PBMCs. Both anti-PD-1 antibody and FMO showed no R1 region. Three samples were overlaid to show reduction of PD-1. FMO indicates Fluorescence Minus One; N, no expression; PBMC, peripheral blood mononuclear cell.

FIG. 2: Identification of 7 Parent and 12 Child Populations; proposed gating strategies for the enumeration of 19 total populations. Mononuclear cells were first gated by SS and CD45. T cells, NK cells, and NKT cells were distinguished by CD3 and CD56. T523 cell subsets were separated by CD4 and CD8, and by memory and naïve. NKT cells were subcategorized by CD4 and CD8. CD3-CD56 cells were subgrouped by CD45 and CD4 to capture B cells, LIN-neg cells, and monocytes. Grans were gated by CD45 and SSC hi. “Grans” indicates granulocytes; LINneg, lineage negative; neg, negative; NK, natural killer; NKT, natural killer T cell; SS, side scatter.

FIG. 3: Distribution of General Populations, CTLA-4-pos Cells, and PD-1-pos Cells. Each of the 7 parent populations were measured as percentage of mononuclear cells (MNCs) except granulocytes, which were measured as a total of CD45-pos cells. Child populations were measured as a percentage of the parent population. CTLA-4-pos and PD-1-pos cells were plotted as a percentage of the specific parent or child population indicated. Box and whiskers plot of each set of values are shown. Asterisk indicates statistical differences compared with the HV cohort. False discovery rate with a set q-value of 10% was used for multiple t-test comparisons. Dotted line indicates even counts too low to analyze. CLL indicates chronic lymphocytic leukemia; GBM, glioblastoma multiforme; HV, healthy volunteer; pos, positive.

FIGS. 4A-4B: HV Compared With Thyroid Cancer Patient 9 (Thy 9). FIG. 4A: HV and Thy 9 samples run on same instrument and same day, and with same master mix. FIG. 4B: Thy 9 showed CTLA-4 expression, which was found to be specific and not in all populations.

FIGS. 5A-5C: Hierarchical Clustering and Profiling of PD-1. FIG. 5A: Hierarchical clustering of PD-1-positive cells was performed on a sample of healthy volunteers and patients with GBM, liver tumor, CLL, and thyroid cancer. B cells were removed from the analysis because of high levels in the CLL cohort. NKT-DP, NKT-CD4, and NKT-DN did not reach minimum event criteria and were not included. Five major profiles clustered, and samples that did not fall into a cluster were removed for clarity. FIG. 5B: Five clusters were identified as 1 (orange), 2 (blue), 3 (green), 4 (purple), and 5 (red). FIG. 5C: PD-1-positive cells of patients in each profile were plotted and a 1-way analysis of variance performed to determine statistical significance between profiles. Data for profile 5 patients are not shown because of extremely high values that did not fit well with graph parameters. mem, memory; monos, monocytes; nai, naïve.

FIG. 6: Leukocyte values of the five profiles found in FIG. 5C were plotted independent of PD-1. The colors designate patient profiles as found in the hierarchical clustering analysis: 1 (orange), 2 (blue), 3 (green), 4 (purple), and 5 (red). A one-way ANOVA was used to determine significance between profiles.

FIG. 7A: Absolute cell counts of patients on pembrolizumab. FIG. 7B: Percentage of cells of parent population for patients on pembrolizumab. Denotation for disease groups is as follows: “CR” clear and complete response, “benefit” patients who achieved a clear partial response, “PD” those who had disease progression at their first disease reassessment. Patients with “questionable benefit” who either achieved a mixed response (progression at some sites with regression or stable disease at other sites) or had clinical benefit that was not clearly related to immunotherapy were not included in the analysis.

FIG. 8: Percentage of cells of parent population for patients on pembrolizumab.

FIG. 9: Hierarchical Clustering and Profiling of HV (n=20) and patients on Pembrolizumab pre-treatment (n=16) and their post-treatment follow up visit. Two profiles were created. Profile 1 consisted of all healthy volunteers, all pre-treatment samples, and one follow up sample. Profile 2 consisted of all follow up samples.

FIGS. 10A and 10B: Hierarchical clustering was performed on pre-treatment values of cancer patients and HV, and two unique profiles were identified. Principle component analysis was used to determine which phenotypes were the greatest contributors to each profile.

FIG. 11: A scoring system was developed based on the single analyte immune phenotyping data, and the scoring system was tested on 16 patients treated with pembrolizumab. Of the patients that benefitted or had a full recovery (filled circles), 9 out of 10 had a score of 1 or greater. For patients that had progressive disease (open circles), 6 out of 6 had scores of zero or lower.

### DETAILED DESCRIPTION

This document provides methods and materials involved in assessing and treating mammals having cancer. Examples of cancers that can be assessed and/or treated as described herein include, without limitation, liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma. For example, this document provides methods and materials for using distinct phenotypes as singular biomarkers (e.g., PD-1⁺CD3⁺ double negative T cells) or combinations of immune phenotypes (classified as immune profiles) to determine whether or not a mammal having cancer is likely to respond to treatment with pembrolizumab. As described herein, an elevated presence of one or more particular immune phenotypes (e.g., as compared to a control level determined in one or more healthy mammals that do not have cancer) can indicate that a mammal is likely to respond to treatment with pembrolizumab. In some cases, a reduced presence of those immune phenotypes or the increased presence of one or more different immune phenotypes (e.g., as compared to a control level determined in one or more healthy mammals that do not have cancer) can indicate that the mammal is less likely to respond to treatment with pembrolizumab. Examples of responses to treatment with pembrolizumab include, for example, reduced tumor size, reduced number of cancer cells in the mammal, and/or reduced tumor growth rate.

The terms “elevated level” and “elevated presence” as used herein with respect to an immune phenotype measured in a biological sample refers to a level of a particular cell type (e.g., a leukocyte or lymphocyte that displays one or more particular markers, such as CD152, CD45RO, CD56, CD3, CD8, CD28, CD4, CD45, PD-1, and/or CTLA-4) that is greater (e.g., at least 5, 10, 25, 35, 45, 50, 55, 65, 75, 80, 90, 100, or more than 100 percent greater) than the median level of that cell type measured in a control biological sample from a control mammal (e.g., a healthy mammal that does not have cancer). Appropriate methods for identifying a biological sample as having an elevated presence of one or more immune phenotypes described herein include, without limitation, flow cytometry-based methods as described herein.

The terms “reduced level” and “reduced presence” as used herein with respect to an immune phenotype measured in a biological sample refers to a level of a particular cell type (e.g., a leukocyte or lymphocyte that displays one or more particular markers, such as CD152, CD45RO, CD56, CD3, CD8, CD28, CD4, CD45, PD-1, and/or CTLA-4) that is less (e.g., at least 5, 10, 25, 35, 45, 50, 55, 65, 75, 80, 90, or more than 90 percent less) than the median level of that cell type measured in a control biological sample from a control mammal (e.g., a healthy mammal that does not have cancer). Appropriate methods for identifying a biological sample as having a reduced presence of one or more immune phenotypes described herein include, without limitation, flow cytometry-based methods as described herein.

The methods and materials provided herein can be used to assess any appropriate mammal having cancer, including, without limitation, humans, non-human primates, horses, cows, sheep, pigs, goats, rabbits, mice, and rats. Further, any appropriate biological sample containing leukocytes and/or lymphocytes can be used in the methods provided herein. Suitable biological samples include, without limitation, blood, peripheral blood mononuclear cells (PBMCs), bone marrow, and lymph.

As described herein, a mammal (e.g., a human) can be identified as being a responder (likely to respond to treatment with pembrolizumab) or a non-responder (not likely to respond to treatment with pembrolizumab), by determining that a biological sample from the mammal has a particular immune phenotype or immune profile. Any suitable method can be used to identify an immune phenotype or immune profile in biological sample from a mammal. In some cases, for example, flow cytometry methods can be used to determine an immune phenotype by measuring the amount or proportion of a particular population of cells (e.g., leukocytes or lymphocytes) that display one or more markers (e.g., PD-1, CTLA-4, CD152, CD45RO, CD56, CD3, CD8, CD28, CD4, CD45, or any combination thereof). An immune profile can include two or more (e.g., two, three, four, five, six, seven, eight, nine, ten, 11, 12, 13, 14, 15, 16, 17, 18, 19, 20, or more than 20) immune phenotypes. Examples of immune phenotypes that can be evaluated using the methods provided herein include, without limitation, those set forth in TABLE 3 herein.

An immune phenotype can be correlated with response or non-response, or can be categorical with a clinical outcome. Phenotypes can have a wide expression range between members of a population, and can be affected by age, sex, natural history, and disease, for example. Examples of immune phenotypes that can be measured and used individually to classify a mammal (e.g., a human) as being a responder or a non-responder include PD1+CD3+ DN (% CD3) (the percentage of CD3+ T cells that also are PD1+ and double negative), PD1+CD8+ Naïve (% CD8), number of Granulocytes (“Grans,” cells/μL), Neutrophils 15+16+ (cells/μL), Grans (% CD45), Eosinophils 15+16− (cells/μL), PD1+CD3+ (% MNC), PD1+CD8+(% CD3), Intermediate Monocytes 14+16+ (cells/μL), CD3 (% MNC), and Monocytes (% CD45pos). Examples of immune phenotypes that can be measured and used individually to classify a mammal (e.g., a human) as being likely to respond to treatment with pembrolizumab include, without limitation, PD1+CD8+ Naïve (% CD8), Grans (cells/μL), Neutrophils 15+16+ (cells/μL), Grans (% CD45), Eosinophils 15+16− (cells/μL), PD1+CD3+ (% MNCs), and PD1+CD8+ (% CD3). That is, the elevated level of any of these immune phenotypes in a biological sample from a mammal can indicate that the mammal is likely to benefit from treatment with pembrolizumab. Examples of immune phenotypes that can be identified and used individually to classify a mammal (e.g., a human) as not being likely to respond to treatment with pembrolizumab include, without limitation, PD1+CD3+ DN (% CD3), number of Intermediate Monocytes 14+16+ (cells/μL), CD3 (% MNCs), and Monocytes (% CD45pos). That is, the elevated level of any of these immune phenotypes in a biological sample from a mammal can indicate that the mammal is not likely to benefit from treatment with pembrolizumab.

Immune profiles are made up of a combination of phenotypes (e.g., the phenotypes disclosed above), typically across a large number of patients. An immune phenotype can be determined using bioinformatics to simultaneously identify phenotypes with similar expression (grouped) across the patient populations, as well as to group patients with similar expressed phenotypes. The similar expressed phenotypes that effectively represent patients with similar immune systems make up an immune profile. Profiles can be used to identify correlation with prognosis, response, survival, etc. Profiles also can be used to identify the major phenotypes that contribute to the profile, and can sort and identify the phenotypes that are most important. Thus, immune profiles can help to identify combinations of phenotypes that together generate powerful prognostic tools.

In some cases, a mammal (e.g., a human) can be identified as being likely to respond to treatment with pembrolizumab, or identified as not being like to respond (or being less likely to respond) to treatment with pembrolizumab, using an algorithm based on scores assigned to a combination of immune phenotypes identified in the mammal. An example of such an algorithm is set forth in TABLE 6 herein. For example, a biological sample (e.g., a peripheral blood sample) from a mammal (e.g., a human) can be evaluated for the single analyte phenotypes listed in TABLE 6. Each phenotype that is detected in the sample can be assigned the score listed in the table, and the individual scores can be summed to arrive at a total score. A total score of 1 or above can identify the mammal as being likely to respond to treatment with pembrolizumab, while a score less than 1 can identify the mammal as not being likely to respond (or being less likely to respond) to treatment with pembrolizumab.

Once a mammal (e.g., a human) is identified as having a biological sample with a particular immune phenotype or immune profile as described herein, the mammal can be classified as being likely to respond to treatment with pembrolizumab, or as not being likely to respond to treatment with pembrolizumab. For example, a human identified as having a biological sample with one or more (e.g., two, three, four, five, six, seven, eight, nine, ten, or more than 10) immune phenotypes described herein as indicating response to pembrolizumab can be classified as being likely respond to pembrolizumab treatment. In some cases, a mammal (e.g., a human) identified as having a biological sample with one or more (e.g., two, three, four, five, six, seven, eight, nine, ten, or more than 10) immune phenotypes described herein as indicating a lack of response to pembrolizumab can be classified as not being likely respond to pembrolizumab treatment.

Any combination of the immune phenotypes disclosed herein can be evaluated. For example, when two or more immune phenotypes are used to identify a mammal as being likely to respond to treatment with pembrolizumab, the two phenotypes can be PD1+CD8+ Naïve (% CD8) and Grans (cells/μL), PD1+CD8+Naïve (% CD8) and Neutrophils 15+16+ (cells/μL), PD1+CD8+ Naïve (% CD8) and Grans (% CD45), PD1+CD8+ Naïve (% CD8) and Eosinophils 15+16− (cells/μL), PD1+CD8+ Naïve (% CD8) and PD1+CD3+ (% MNCs), PD1+CD8+ Naïve (% CD8) and PD1+CD8+ (% CD3), Grans (cells/μL) and Neutrophils 15+16+ (cells/μL), Grans (cells/μL) and Grans (% CD45), Grans (cells/μL) and Eosinophils 15+16− (cells/μL), Grans (cells/μL) and PD1+CD3+ (% MNCs), Grans (cells/μL) and PD1+CD8+ (% CD3), Neutrophils 15+16+ (cells/μL) and Grans (% CD45), Neutrophils 15+16+ (cells/μL) and Eosinophils 15+16− (cells/μL), Neutrophils 15+16+ (cells/μL) and PD1+CD3+ (% MNCs), Neutrophils 15+16+ (cells/μL) and PD1+CD8+ (% CD3), Grans (% CD45) and Eosinophils 15+16− (cells/μL), Grans (% CD45) and PD1+CD3+ (% MNCs), Grans (% CD45) and PD1+CD8+ (% CD3), Eosinophils 15+16− (cells/μL) and PD1+CD3+ (% MNCs), Eosinophils 15+16− (cells/μL) and PD1+CD8+ (% CD3), and PD1+CD3+ (% MNCs) and PD1+CD8+(% CD3).

When two or more immune phenotypes are used to identify a mammal as not being likely to respond to treatment with pembrolizumab, the two phenotypes can be PD1+CD3+ DN (% CD3) and Intermediate Monocytes 14+16+ (cells/μL), PD1+CD3+ DN (% CD3) and CD3 (% MNCs), PD1+CD3+ DN (% CD3) and Monocytes (% CD45pos), Intermediate Monocytes 14+16+ (cells/μL) and CD3 (% MNCs), Intermediate Monocytes 14+16+ (cells/μL) and Monocytes (% CD45pos), and CD3 (% MNCs) and Monocytes (% CD45pos). Similarly, any combination of three, four, five, six, or all seven of the immune phenotypes described herein can be evaluated to determine whether a mammal is likely to respond to pembrolizumab treatment, or whether the mammal is not likely to respond to pembrolizumab treatment.

This document also provides methods and materials for treating a mammal identified as being likely to respond to pembrolizumab. Any appropriate mammal identified as being likely to respond can be treated with pembrolizumab. This document also provides methods and materials for treating a mammal identified as not being likely to respond to pembrolizumab, where the mammal is treated with a therapeutic other than pembrolizumab (e.g., standard chemotherapy, radiation, or one or more alternative checkpoint inhibitors, such as nivolumab, cemiplimab, atezolizumab, avelumab, durvalumab, ipilimumab, or any combination thereof). Any appropriate mammal identified as not being likely to respond to pembrolizumab can be treated with an alternate to pembrolizumab. Having the ability to identify mammals who are or are not likely to respond to treatment with pembrolizumab can allow clinicians and patients to proceed with treatment options that more effectively treat the cancer.

For example, in some cases, if a mammal (e.g., a human) is identified as not being likely to respond to treatment with pembrolizumab, the mammal can be treated with nivolumab. The nivolumab can be administered at any appropriate dose, at any appropriate frequency, and for any appropriate duration, as described herein. For example, a method provided herein can include administering nivolumab to a mammal identified as not being likely to respond to treatment with pembrolizumab, where the nivolumab is administered at a dose of about 50 mg to about 600 mg, at a frequency from about once a day to about once a month, for a duration ranging from weeks to years (e.g., two to four weeks, one to two months, two to four months, four to six months, six to 12 months, one to two years, two to three years, or more than three years).

In some cases, if a mammal (e.g., a human) is identified as not being likely to respond to treatment with pembrolizumab, the mammal can be treated with cemiplimab. The cemiplimab can be administered at any appropriate dose, at any appropriate frequency, and for any appropriate duration, as described herein. For example, a method provided herein can include administering cemiplimab to a mammal identified as not being likely to respond to treatment with pembrolizumab, where the cemiplimab is administered at a dose of about 50 mg to about 600 mg, at a frequency from about once a day to about once a month, for a duration ranging from weeks to years (e.g., two to four weeks, one to two months, two to four months, four to six months, six to 12 months, one to two years, two to three years, or more than three years).

In some cases, if a mammal (e.g., a human) is identified as not being likely to respond to treatment with pembrolizumab, the mammal can be treated with atezolizumab. The atezolizumab can be administered at any appropriate dose, at any appropriate frequency, and for any appropriate duration, as described herein. For example, a method provided herein can include administering atezolizumab to a mammal identified as not being likely to respond to treatment with pembrolizumab, where the atezolizumab is administered at a dose of about 50 mg to about 600 mg, at a frequency from about once a day to about once a month, for a duration ranging from weeks to years (e.g., two to four weeks, one to two months, two to four months, four to six months, six to 12 months, one to two years, two to three years, or more than three years).

In some cases, if a mammal (e.g., a human) is identified as not being likely to respond to treatment with pembrolizumab, the mammal can be treated with avelumab. The avelumab can be administered at any appropriate dose, at any appropriate frequency, and for any appropriate duration, as described herein. For example, a method provided herein can include administering avelumab to a mammal identified as not being likely to respond to treatment with pembrolizumab, where the avelumab is administered at a dose of about 50 mg to about 600 mg, at a frequency from about once a day to about once a month, for a duration ranging from weeks to years (e.g., two to four weeks, one to two months, two to four months, four to six months, six to 12 months, one to two years, two to three years, or more than three years).

In some cases, if a mammal (e.g., a human) is identified as not being likely to respond to treatment with pembrolizumab, the mammal can be treated with durvalumab. The durvalumab can be administered at any appropriate dose, at any appropriate frequency, and for any appropriate duration, as described herein. For example, a method provided herein can include administering durvalumab to a mammal identified as not being likely to respond to treatment with pembrolizumab, where the durvalumab is administered at a dose of about 50 mg to about 600 mg, at a frequency from about once a day to about once a month, for a duration ranging from weeks to years (e.g., two to four weeks, one to two months, two to four months, four to six months, six to 12 months, one to two years, two to three years, or more than three years).

In some cases, if a mammal (e.g., a human) is identified as not being likely to respond to treatment with pembrolizumab, the mammal can be treated with ipilimumab. The ipilimumab can be administered at any appropriate dose, at any appropriate frequency, and for any appropriate duration, as described herein. For example, a method provided herein can include administering ipilimumab to a mammal identified as not being likely to respond to treatment with pembrolizumab, where the ipilimumab is administered at a dose of about 50 mg to about 600 mg, at a frequency from about once a day to about once a month, for a duration ranging from weeks to years (e.g., two to four weeks, one to two months, two to four months, four to six months, six to 12 months, one to two years, two to three years, or more than three years).

In some cases, an effective dose of pembrolizumab or another therapeutic agent can be administered to a mammal having cancer once or multiple times over a period of time ranging from days to months. Effective doses can vary depending on the severity of the cancer, the route of administration, the age and general health condition of the subject, excipient usage, the possibility of co-usage with other therapeutic treatments, and the judgment of the treating physician. An effective amount of pembrolizumab or another therapeutic agent can be any amount that reduces the likelihood that the cancer will progress, or any amount that reduces disease symptoms (e.g., tumor size or cancer cell number), or any amount that prolongs survival (e.g., overall survival or progression-free survival) without producing significant toxicity to the mammal. For example, an effective amount of pembrolizumab can be from about 50 mg to about 600 mg (e.g., from about 50 to about 100 mg, from about 100 to about 200 mg, from about 200 to about 300 mg, from about 300 to about 400 mg, from about 400 to about 500 mg, or from about 500 to about 600 mg).

The frequency of administration of pembrolizumab or another therapeutic agent to a mammal having cancer can be any frequency that reduces the symptoms of the cancer, reduces the likelihood that the cancer will progress, or increases survival (e.g., overall survival or progression-free survival) of the mammal without producing significant toxicity to the mammal. For example, the frequency of administration can be from about once a day to about once a month (e.g., from about once a week to about once every other week, from about once every other week to about once every three weeks, from about once every three weeks to about once every four weeks, from about once every four weeks to about once every six weeks, from about once every six weeks to about once every other month, about once a week, about once every two weeks, about once every three weeks, about once every four weeks, about once every five weeks, about once every six weeks, or about once every eight weeks). The frequency of administration can remain constant or can be variable during the duration of treatment. In some cases, a course of treatment with pembrolizumab or another therapeutic agent can include rest periods. For example, pembrolizumab can be administered weekly over a two- to four-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition may require an increase or decrease in administration frequency.

An effective duration for administering a composition containing pembrolizumab or another therapeutic agent to a mammal having cancer can be any duration that alleviates one or more symptoms of the cancer, reduces the likelihood that the cancer will progress, or increases survival (e.g., overall survival or progression-free survival) of the mammal, without producing significant toxicity to the mammal. In some cases, the effective duration can vary from weeks to months to years. Multiple factors can influence the actual effective duration used for a particular treatment. For example, an effective duration can vary with the frequency of administration, effective amount, use of multiple treatment agents, route of administration, and severity of the condition being treated.

The invention will be further described in the following examples, which do not limit the scope of the invention described in the claims.

### EXAMPLES

### Example 1—Materials and Methods

Patients and Healthy Volunteers (HVs)

Peripheral blood was collected from 60 HVs and 20 patients with GBM, 10 with liver tumors (6 primary liver tumors and 4 liver metastases), 9 with CLL, and 35 with thyroid cancer. GBM patients were actively participating in a dendritic cell vaccine trial, and had temozolomide, surgery, and radiation treatment before sample collection. CLL patients were newly diagnosed and had no treatment. Liver tumor patients' samples were collected at baseline, prior to the start of SBRT treatment (Delivanis et al., J Clin Endocrinol Metab 2017; 102: 2770-2780; and Gustafson et al., Adv Rad Oncol 2017; 2: 540-547). The thyroid cancer group consisted of patients without active disease, with advanced but stable disease, and with advanced disease soon to start its treatment. Peripheral blood also was collected from a separate group of 26 cancer patients (21 lung, 4 melanoma, and 1 genitourinary) prior to being treated with pembrolizumab or atezolizumab or other checkpoint inhibitor treatment combinations and several time points following (TABLE 2).

Whole Blood Flow Cytometry and Gating Strategies

Whole blood was stained on the day of collection using an established method for identifying multiple cell populations by flow cytometry, along with sources for antibodies and instrument settings, and analysis methods that capture data on more than 120 phenotypes. The staining protocol, instrument settings, reagents, and manufacturers' details were used as described elsewhere (Gustafson et al., PLoS One 2015; 10: e0121546). A 7 tube panel was used to identify a variety of leukocyte populations, including TBNK lyse no wash, T cell-1, B cell, Myeloid, and Granulocyte populations (Gustafson et al. 2015, supra), as well as Monocytes and T cell-2 populations (Gustafson et al., J Immunother Cancer 2017; 5: 30), although only T cell-2 and TBNK assays were used for the analyses described herein. Briefly, 100 μL of fresh whole blood was blocked for 5 minutes with 50 μL of mouse serum (Sigma-Aldrich, St. Louis, MO, Cat #M5905) and stained with the appropriate antibodies for 15 minutes in the dark at room temperature. Following staining, samples were lysed for 20 minutes with 2 mL of Versa-Lyse reagent (Beckman Coulter, Indianapolis, IN, Catalog #A09777), centrifuged for 5 minutes at 1500 RPM, washed with PBS-FE (PBS (Gibco, Catalog #14190) containing 1% albumin (Sigma Aldrich, St. Louis, MO, Catalog #A7034) and 5 mM EDTA (Sigma Aldrich, Catalog #E7889), and fixed in 1% paraformaldehyde. The wash step was not performed on the TBNK assay, but rather 100 μL Flow Count Fluorospheres (Beckman Coulter, Catalog #B96656) were added directly and the sample was collected immediately on the flow cytometer. All samples were run on a 3-laser 10-color Gallios flow cytometer (Beckman Coulter; Chaska, MN).

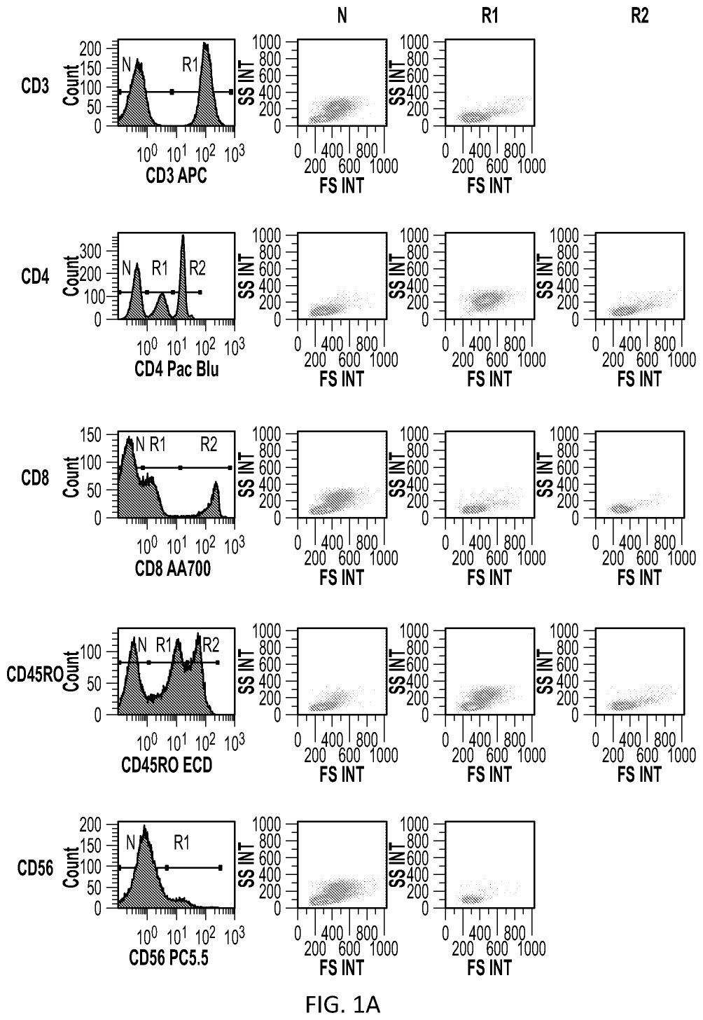

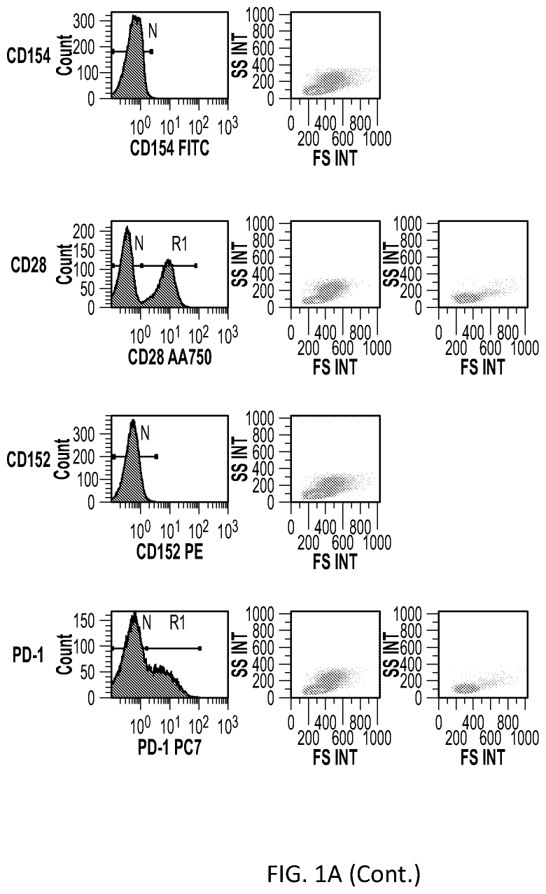

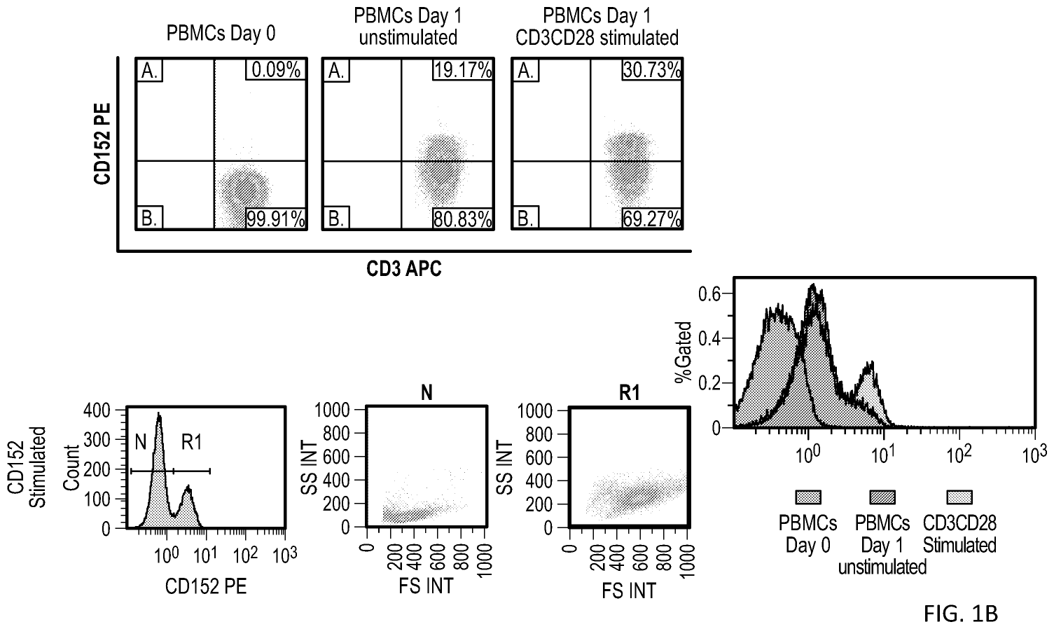

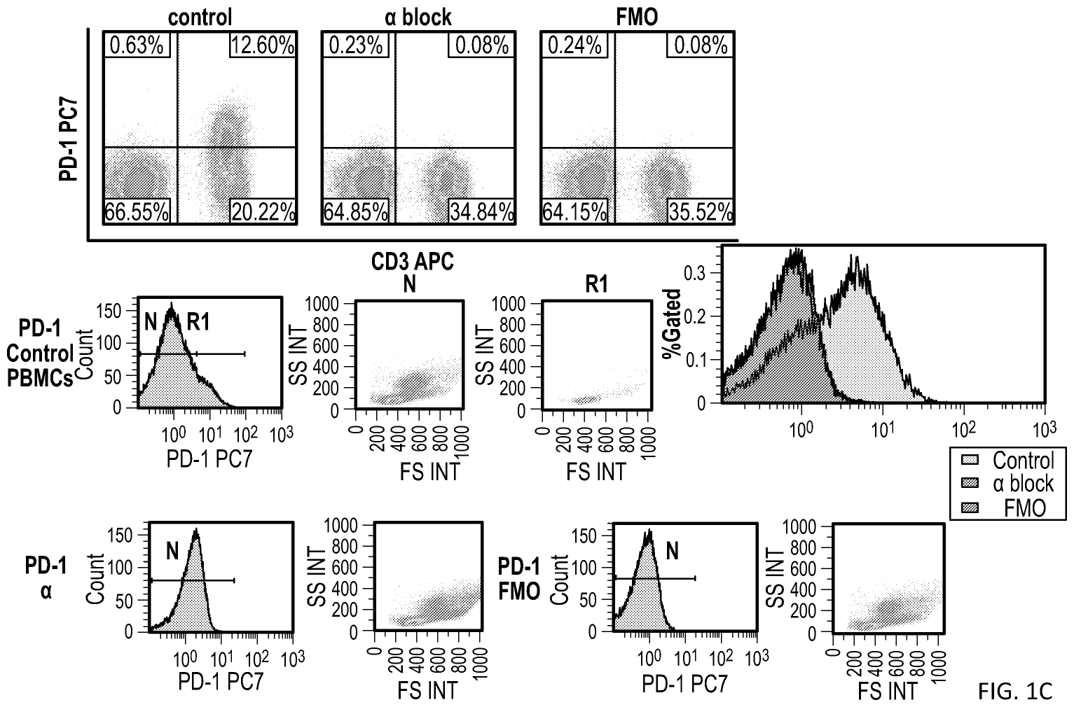

An extended analysis focused on T-cell phenotypes was performed using the markers PD-1, CTLA-4, CD152, CD45RO, CD56, CD3, CD8, CD28, CD4, and CD45 (TABLE 3). T-cell parent populations were characterized by side scatter, forward scatter, and CD45+, CD3+, CD4+, CD8+, and CD4+/CD8⁺ subpopulations. These populations were assessed for PD-1 and CTLA-4 positivity. Cell populations not meeting the minimum criteria of 100 events were excluded from PD-1 and CTLA-4 analyses. For histogram analyses, a HV whole blood sample was stained with the T-cell signaling panel. Histograms were generated from each of the 10 antibodies (except CD45) and used to delineate mononuclear populations (defined by CD45⁺ SSC lo/med) (FIG. 1A). Regions (R1 and R2) were designated for each peak of expression, including peaks with no expression (N), for each antibody. Forward scatter and side scatter density plots were created for each histogram peak.

Validation of the CD152 PE antibody was performed with isolated peripheral blood mononuclear cells (PBMCs). Cells were divided into stimulated and unstimulated fractions. Unstimulated cells were analyzed immediately, and stimulated PBMCs were cultured with CD3CD28 DynaBeads (ThermoFisher Scientific, Waltham, Massachusetts) for 24 hours to increase levels of CD152 before they were analyzed (FIG. 1B). The gating strategy was determined with use of the positive CD152 population. PD-1 was similarly validated. Two conditions were used. In the first, PBMCs were blocked with anti-PD-1 antibody before staining. For the second condition, Fluorescence Minus One for PD-1 was performed (FIG. 1C). Both samples were used to set up the gating strategy for PD-1.

Statistical Analysis

Prism version 7.0 (GraphPad Software, Inc.; La Jolla, California) was used for the multiple t-test comparisons and graphical representations. The approach of false discovery rate was used for the multiple t-test comparisons with a 2-stage step-up method described elsewhere (Benjamini et al., Biometrika 2006; 93: 491-507) and a set false discovery rate q-value of 10%. Multiparameter analysis and hierarchical clustering were completed as described elsewhere (Gustafson et al., PLoS One 2017; 12: e0182002). Hierarchical clustering of PD-1 expression was performed with Partek Genomics Suite 6.6 (Partek Inc.; St. Louis, MO). All phenotypes were analyzed except those with event counts less than 100 in PD-1 and CTLA-4 analyses (NKT-DP, NKT-CD4+, and NKT-DN). B-cell counts also were excluded because they would artificially skew clustering due to high levels in the CLL cohort. The analysis was completed as described elsewhere (Gustafson et al., J Immunother Cancer 2013; 1: 7). Statistical significance of the group distribution was verified with Fisher exact test. Significance between profiles was determined with a 1-way analysis of variance.

### Example 2—Confirmation of CTLA-4 and PD-1 Phenotypes and Gating

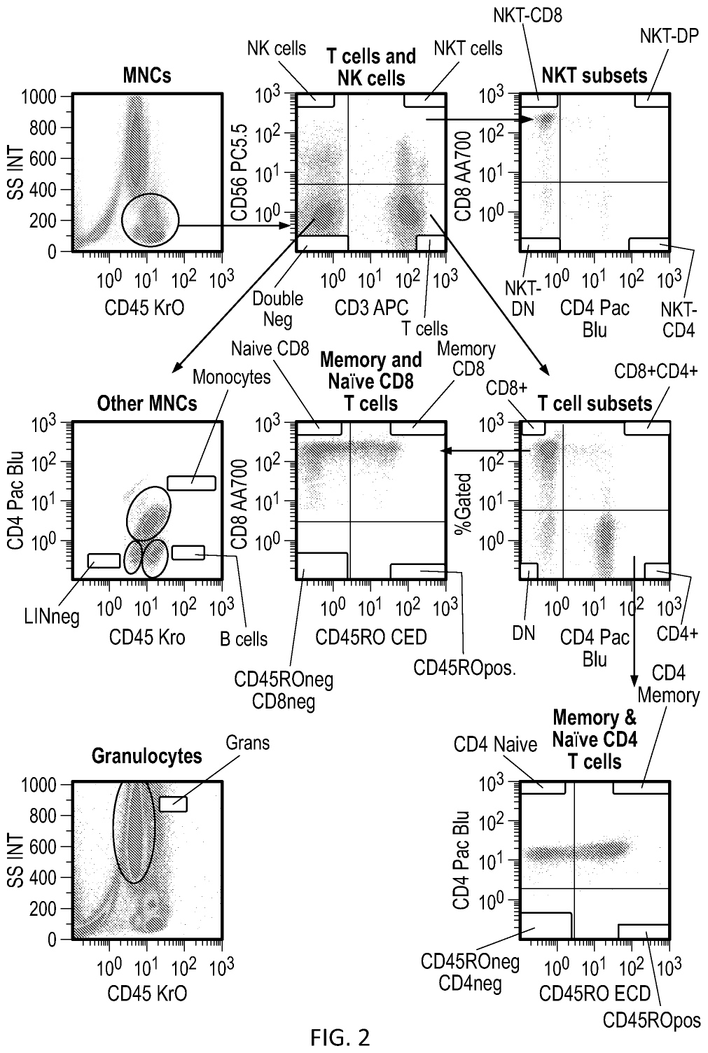

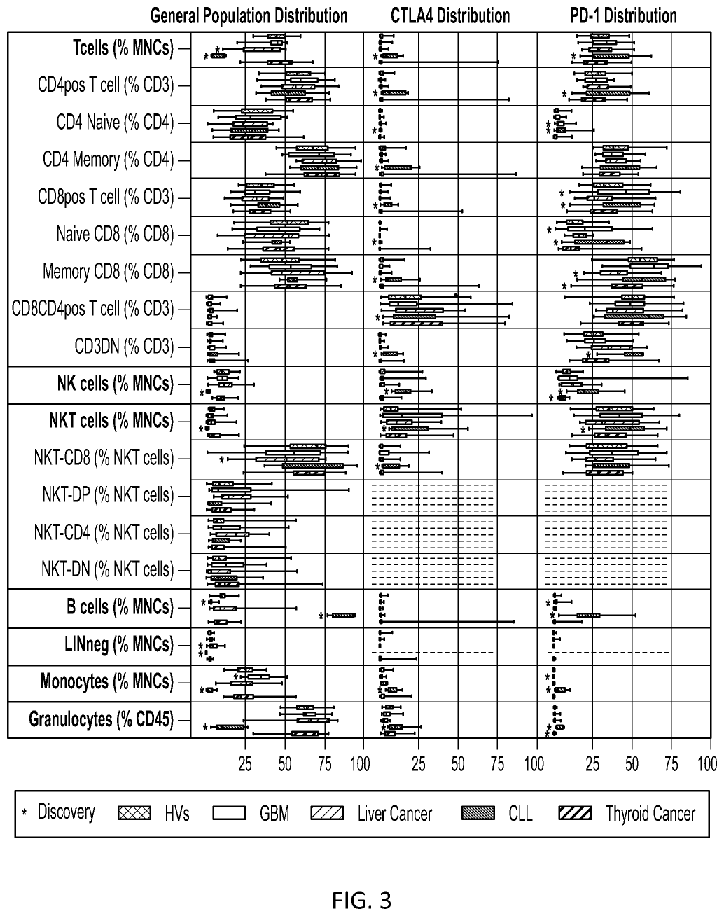

The strategy used in the studies described herein was based on quantitative unbiased assessment of the immune system. The approach used 10 color multi-tube quantitative flow cytometry on whole blood, aimed at identifying the differences of parent (major) and child (minor) populations among the cohorts before specifically looking at PD-1 and CTLA-4. The 7 parent and 12 child populations were gated (FIG. 2 and TABLE 1). Each parent population was measured as a percentage of mononuclear cells and each child population as a percentage of the parent population, with the exception of granulocytes, which were measured as a percentage of CD45-positive cells. Percentages of the populations from each cancer cohort were compared with the HV cohort using a Two-stage linear step-up procedure (Benjamini et al., supra) to identify discoveries for possible biomarkers (FIG. 3). To meet the criteria for a discovery, the difference between the HV group and cohort being compared had to reach a minimum P-value of <0.05 and a FDR (false discovery rate) q-value of <0.10. The results of these studies revealed that the percentage of T cells in both the liver tumor group and the chronic lymphocytic leukemia (CLL) group were less than the percentages in the HV group. Percentages of both natural killer cells (NKs) and natural killer-like T cells (NKTs) were lower in the CLL cohort, and NKT-CD8⁺ cells were lower in the liver tumor cohort. The CLL group had a higher percentage of B cells than the HV group, but had lower lineage-negative cells, monocytes, and granulocytes. Patients with glioblastoma multiforme (GBM) had fewer B cells; patients with liver tumors had more lineage-negative cells.

### Example 3—Identification of White Blood Cell CTLA-4- and PD-1-Positive Staining Populations in HVs and Cancer Patients

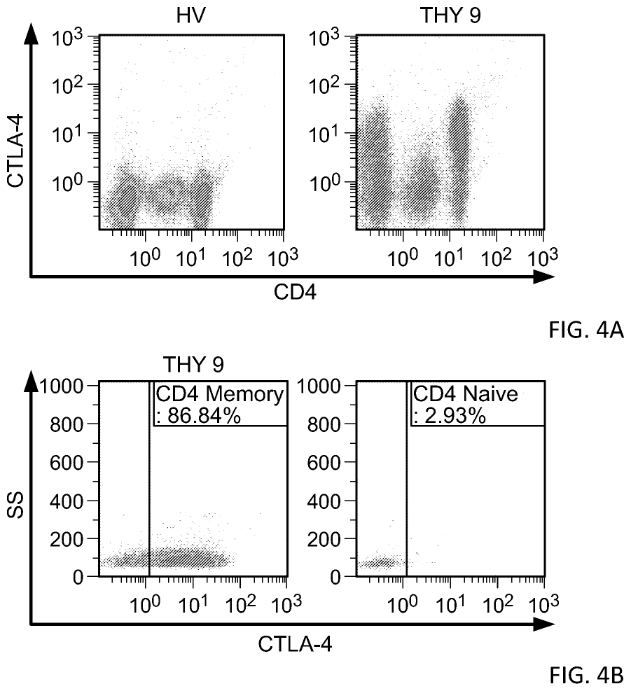

Each population was plotted by side scatter vs CD152 or PD-1 to determine the percentage of cells positive for each marker. To reduce false-positive results and exclude noise, populations that had 100 events or more in the positive gates were evaluated. NKT CD4⁺CD8+(Double Positive, DP), NKT-CD4+, and NKT CD4⁻CD8⁻ (Double Negative, DN) had too few events to include in this analysis. CTLA-4-positive or PD-1-positive populations that were not present in the HVs or were statistically different compared with HV populations (FIG. 3 and TABLE 1) were identified. CTLA-4+DP T cells and CTLA-4⁺ NKT-CD8⁺ cells also were identified; these had not previously been identified. Uniquely, one thyroid cancer patient was found to be an outlier with CTLA-4 positive cells, while all other samples showed no indication of CTLA-4 on the measured populations (FIG. 4).

### Example 4—Hierarchical Clustering of PD-1

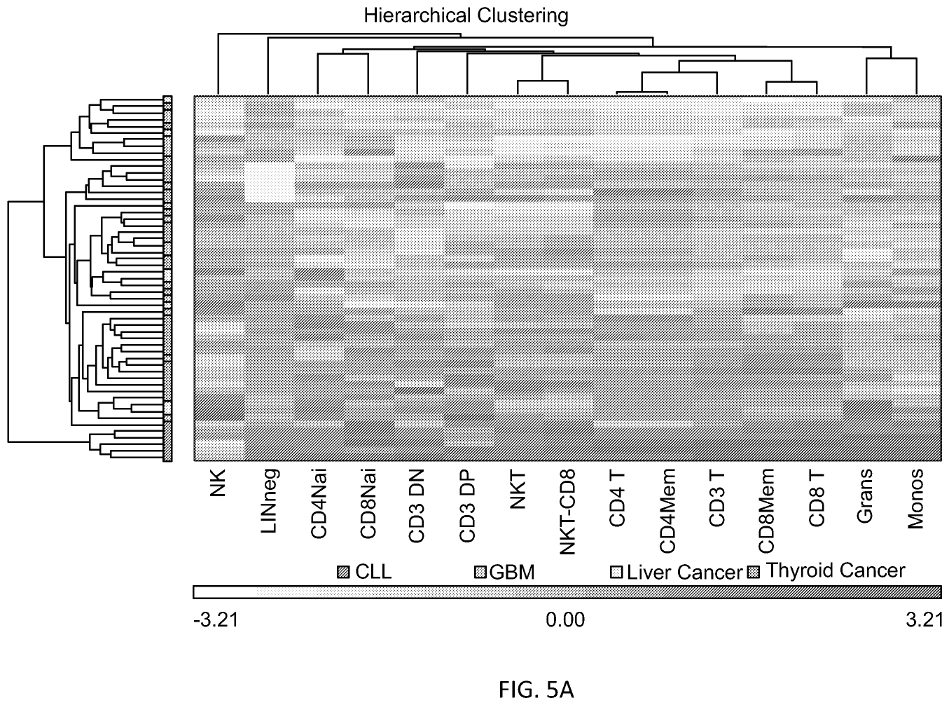

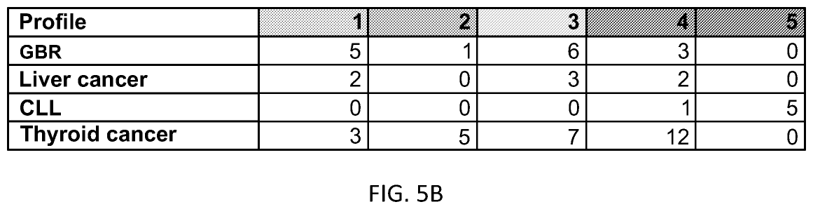

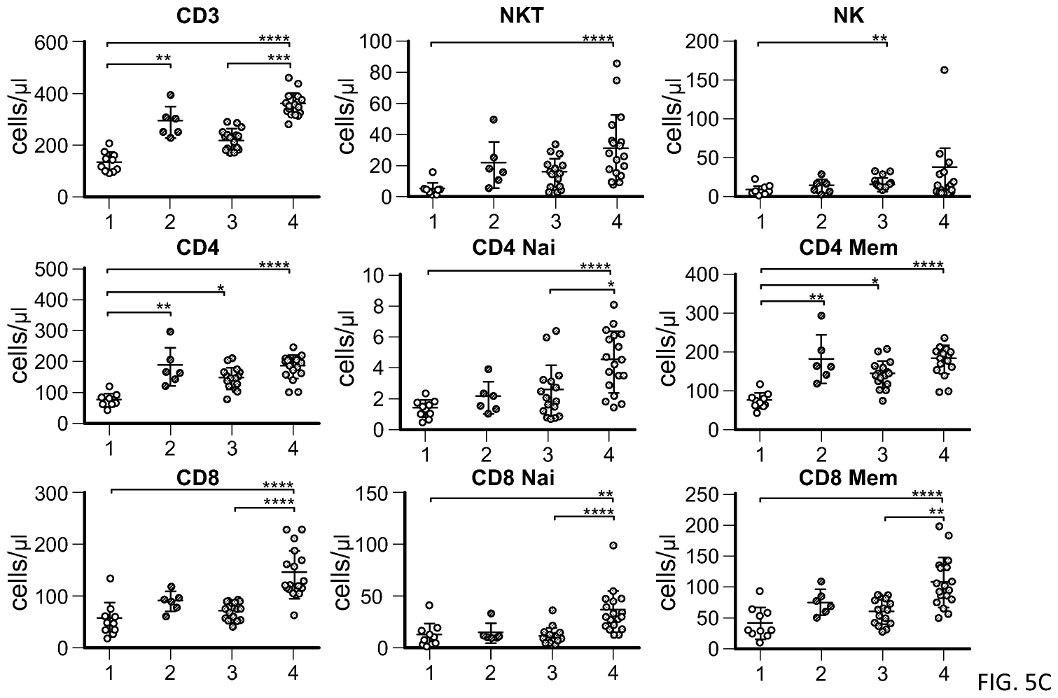

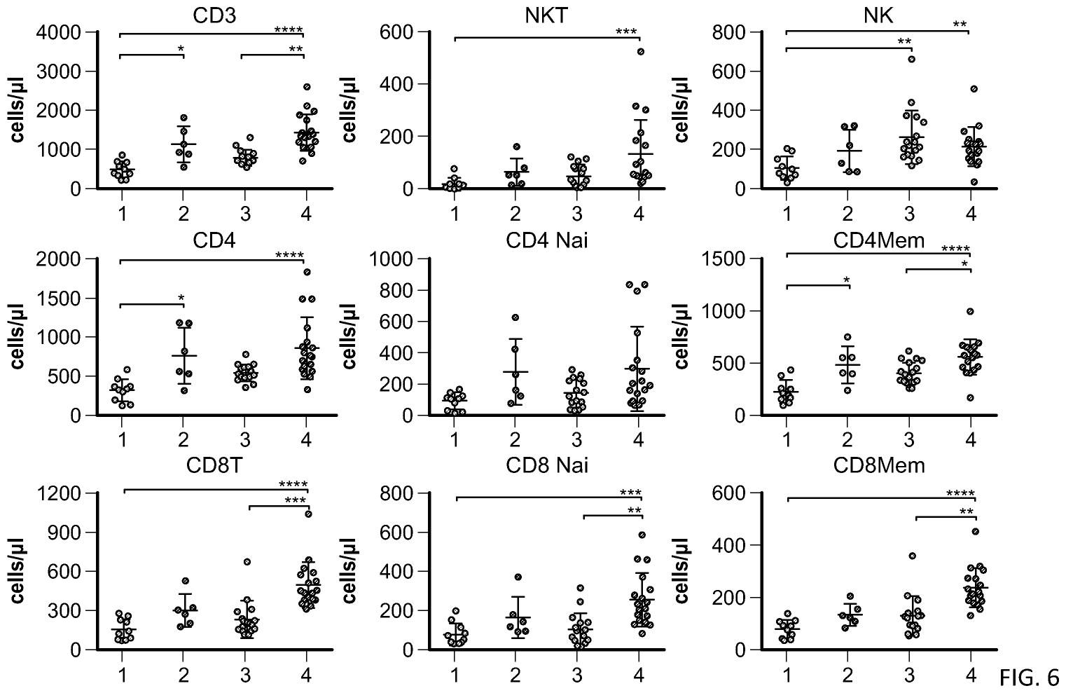

Unsupervised hierarchical clustering has been used to group patients into immune profiles that have correlated with survival in patients diagnosed with cancer and ALS (Gustafson et al., J Immunother Cancer 2013; 1: 7; and Gustafson et al., PLoS One 2017; 12: e0182002). The immune profiles—compositions of all characteristics of the leukocyte populations in their entirety—were influenced but not determined by the cancer type. Thus, immune profiles of patients may be quite heterogeneous within a specific disease entity, but also may be similar and/or shared with patients of a different disease. By clustering these values, five distinct PD-1 profiles were identified (FIG. 5). Clustering was not performed on CTLA-4 because of low values of expression in circulating blood cells. Profiles with fewer than 5 patients were not used for further analysis. Profile 1 consistently had the least amount of cells positive for PD-1 in all 19 populations. Profile 5 was composed of 5 CLL patients with high levels of PD-1 across all populations, making it uniquely characteristic of CLL. Profiles 2 and 3 differed in PD-1-positive cells on the basis of populations, whereas in profile 4, the number of PD-1-positive cells was consistently increased. An advantage of this approach is that it can determine if cancer source (tumor type) determines the evolution of patient's immunity. It was noted that solid tumors were spread among the immune profiles. The unique nature of profile 5 (the only leukemic profile; CLL) was likely due to the influence of the changes of other leukocytes in circulation (i.e., reduction in monocytes, T cells, and granulocytes). Importantly, these data strongly suggested that there is a finite number of immune profiles that describe PD-1 expression across solid tumors, and that methods can be applicable across tumor types without having to repeat studies for each tumor. To verify that these observations did not depend on the leukocyte counts but rather on PD-1, the same analysis was performed using the results of a TBNK assay (T cells, B cells, and NK cells) described elsewhere (Gustafson et al., PLoS One 2015; 10: e0121546), which provided the ability to look at the same populations independent of PD-1 levels. This analytical approach did not cluster the patients into the same profiles as those based on PD-1 (FIG. 6).

### Example 5—Comparison of Patients Receiving Pembrolizumab

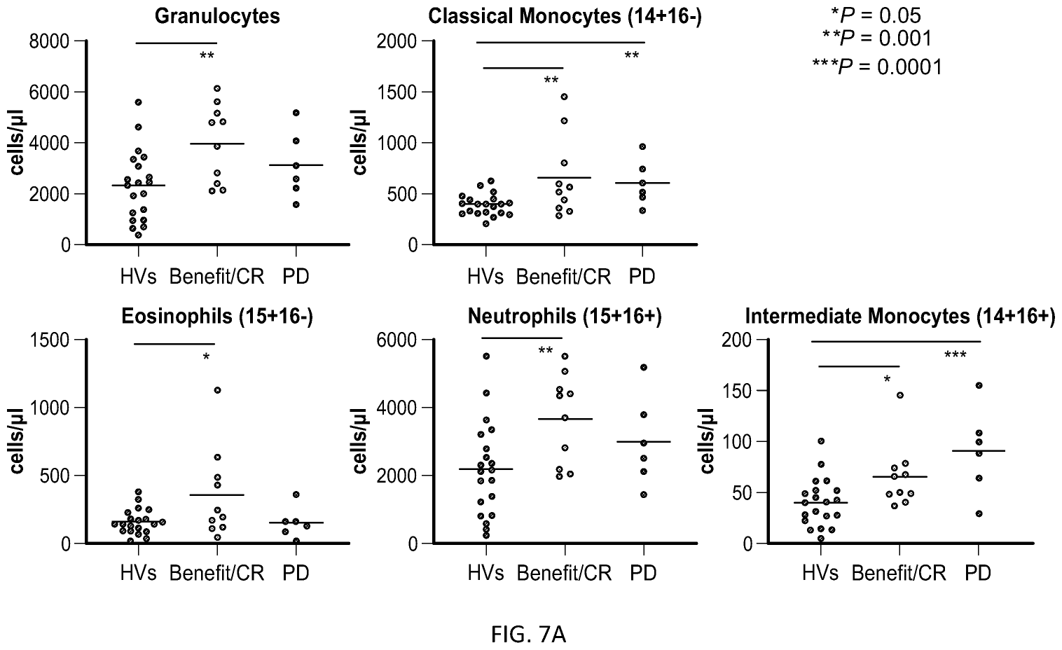

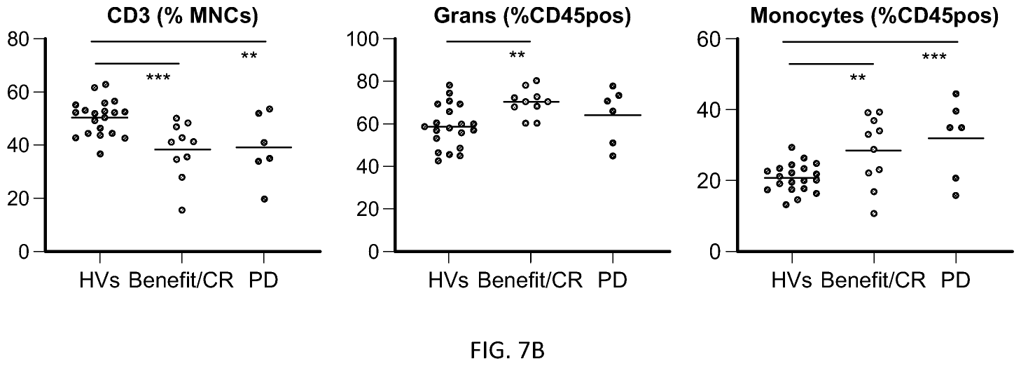

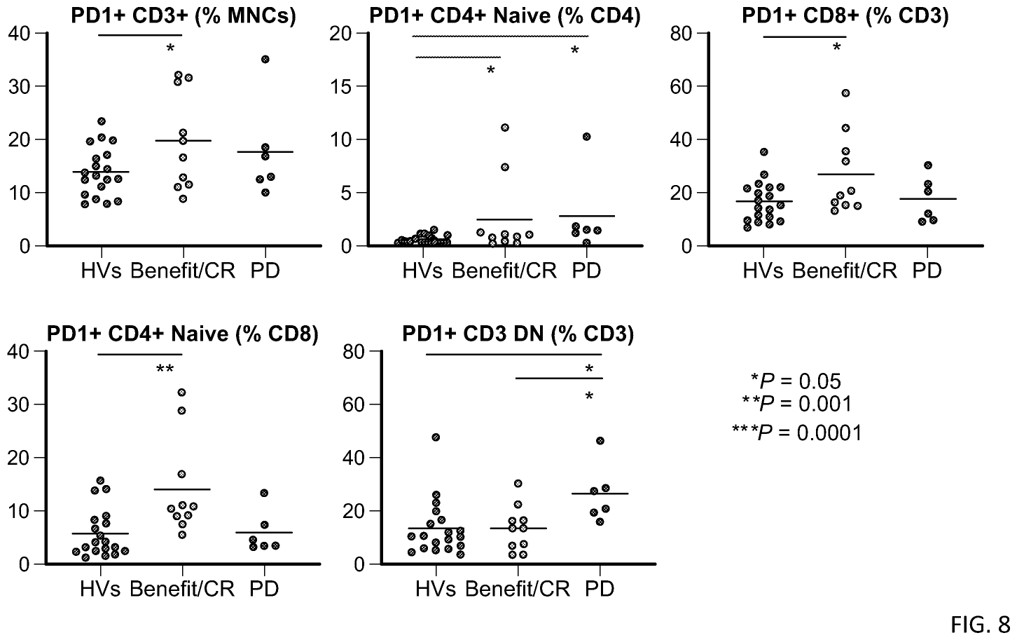

To determine whether phenotypic differences could be identified among responders and non-responders to checkpoint inhibitors, pre-treatment blood samples were collected from 16 patients on Pembrolizumab. Samples were analyzed using both a multi-parameter flow assay to measure PD-1 levels on 19 leukocyte populations as well as the standard TBNK assay. Absolute counts of granulocytes, classical monocytes, neutrophils, and intermediate monocytes were increased (P<0.05) in patients with benefit/complete response (CR) when compared to HV. Similarly, absolute counts of intermediate and classical monocytes showed the same pattern (P≤0.01) in patients with progressive disease (PD) as compared to HV (FIG. 7A). CD3 (% MNCs) were lower (P≤0.01) in benefit/CR and PD groups than in HVs, while both granulocytes (% CD45 positive) and monocytes (% CD45 positive) were increased (P≤0.01) (FIG. 7B). Patients with benefit/CR expressed more PD-1 positive T cells (PD-1⁺CD3⁺, PD-1⁺ Naïve CD4+, PD-1⁺CD8⁺, PD-1⁺ Naïve CD8⁺, PD-1⁺CD3⁺ DN) than HVs. PD-1⁺CD3⁺ DN and PD-1⁺ Naïve CD4⁺ T cells were significantly higher (P≤0.05) in patients with PD than those who received benefit or had complete response, and also compared to HV (FIG. 8). CD3⁺ DN cells, independent of PD-1, have been associated with systemic inflammation (Brandt and Hedrich, Autoimmunity Rev 2018; 17: 422-430), but PD-1⁺CD3⁺ DN cells have not been studied in cancer research or identified as a biomarker for progression of disease. It is possible that many of the CD3⁺ DN cells could be γδ T cells, which are known to increase in PD-1 due to antigenic stimulation (Iwasaki et al., Eur J Immunol 2011; 41: 345-355; and Hoeres et al., Oncoimmunol 2019; 8: 1550618). It is noted that in the studies described herein, patients receiving Pembrolizumab whose disease had progressed also had greater surface expression of PD-1 than the CR or HV groups.

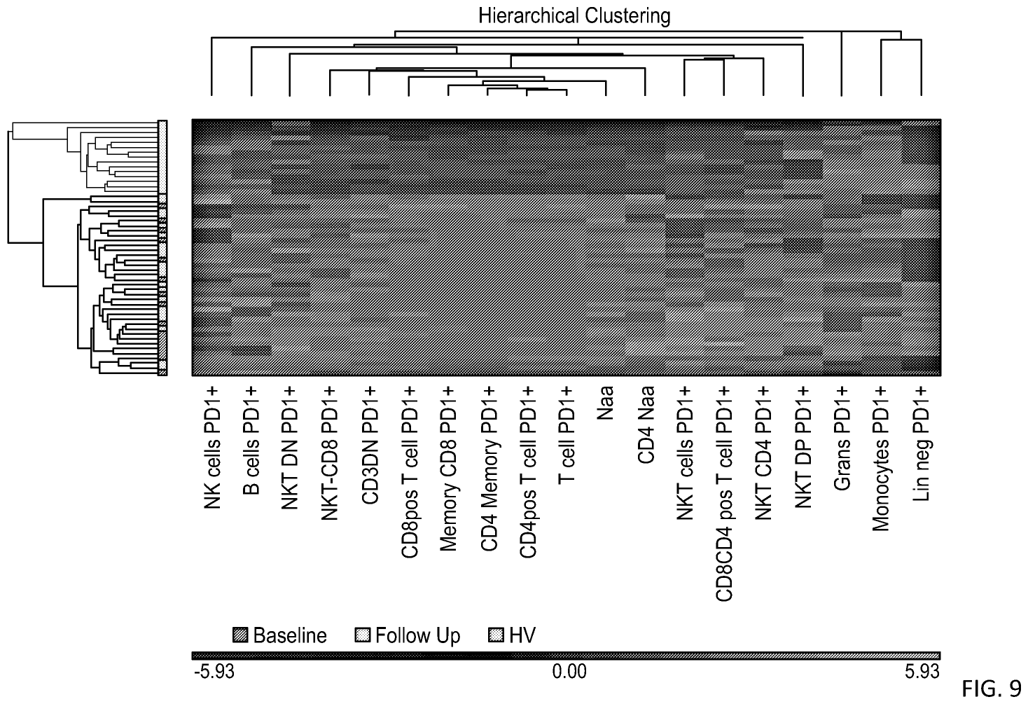

Analysis of single immune phenotypes as predictive biomarkers is fundamentally limited, as rarely do a single population of leukocytes predict immune response. Hierarchical clustering therefore was used to identify patients with similar immune profiles to reveal changes pre-treatment and post treatment that represent global changes in immunity. Patient samples were clustered pre-treatment (n=16) with their correlating post Pembrolizumab treatment follow up sample (n=16), as well as 20 HVs. When all three groups were combined and clustered, two profiles were created. Profile one consisted of all baseline samples, HVs, and one follow up, while profile two consisted purely of follow up samples (FIG. 9). This approach captured the immunological changes caused by the treatment of Pembrolizumab. Reduction of PD-1 by Pembrolizumab was responsible for clustering of post-treatment samples.

TABLE 1
CTLA-4 and PD-1% Values by Cohort
Cohort^{b}

| | | | | | | |
|---|---|---|---|---|---|---|
| | | | Liver | | Thyroid | |
| Population^{a} | HV | GBM | Cancer | CLL | Cancer | Literature^{c, d} |

Mean % CTLA-4-Pos Cells

| | | | | | | |
|---|---|---|---|---|---|---|
| T cells, % MNCs | 1.82 | 1.26 | 1.14 | 6.76^{e} | 3.95 | Fife and Bluestone |
| CD4-pos T cells, % CD3 | 2.19 | 1.04 | 1.34 | 8.75^{e} | 4.48 | Kaufmann et al. |
| CD4 naïve, % CD4 | 0.32 | 0.28 | 0.95 | 0.51^{e} | 0.52 | Jago et al. |
| CD4 memory, % CD4 | 3.09 | 1.28 | 1.5 | 11.46^{a} | 5.24 | Jago et al. |
| CD8-pos T cells, % CD3 | 1.23 | 1 | 0.94 | 4.86^{a} | 2.87 | Long et al. |
| Memory CD8, % CD8 | 2.09 | 1.33 | 1.15 | 8.67^{e} | 3.87 | Long et al. |
| Naïve CD8, % CD8 | 0.33 | 0.43 | 0.15 | 0.55^{e} | 1.43 | Long et al. |
| CD3DN, % CD3 | 0.71 | 0.46 | 0.76 | 6.59^{e} | 1.37 | Anand et al. |
| CD8CD4-pos T cells, % CD3 | 17.96 | 18.31 | 22.37 | 26.54^{e} | 25.94 | — |
| NK cells, % MNC | 2.87 | 2.98 | 2.85 | 16.03^{e} | 2.22 | Beldi-Ferchiou |
| | | | | | | Caillat-Zucman |
| NKT cells, % MNC | 10.52 | 21.86 | 14.32 | 19.06^{e} | 11.67 | Yamagiwa et al. |
| NKT-CD8, % NK T cells | 1.72 | 3.94 | 2.39 | 7.35^{e} | 3.48 | — |
| B cells, % MNC | 1.16 | 0.71 | 0.58 | 0.89 | 3.69 | Steiner et al. |
| LIN neg, % MNC | 0.54 | 0.3 | 0.11 | . . . | 1.2 | Liu et al. |
| Monocytes, % MNC | 2.42 | 1.49 | 2.58 | 8.02^{e} | 2.48 | Wang et al. |
| Granulocytes, % CD45 pos | 6.56 | 4.96 | 3.71 | 11.44^{e} | 6.56 | Fujiwara et al. |

Mean % PD-1-Pos Cells

| | | | | | | |
|---|---|---|---|---|---|---|
| T cells, % MNC | 29.51 | 33.05 | 30.23 | 36.50^{e} | 28 | Fife and Bluestone |
| CD4-pos T cells, % CD3 | 27.8 | 27.29 | 30.49 | 33.37^{e} | 26.84 | Yang et al. |
| CD4 naïve, % CD4 | 2.5 | 2.65 | 5.01^{b} | 6.05^{e} | 2.15 | Pardoll |
| CD4 memory, % CD4 | 39.95 | 38.39 | 39.13 | 43.22^{e} | 36.12 | Foldi et al. |
| CD8-pos T cells, % CD3 | 33.92 | 45.46^{e} | 30.54 | 43.83^{e} | 31.72 | Shi et al. |
| Naïve CD8, % CD8 | 14.61 | 25.53^{e} | 16.38 | 29.19^{e} | 13.97 | Waki et al. |
| Memory CD8, % CD8 | 55.19 | 61.58 | 40.54^{e} | 55.48 | 47.61^{e} | Waki et al. |
| CD8CD4-pos T cells, % CD3 | 49.26 | 49.86 | 44.25 | 53.49 | 48.55 | Chauhan et al. |
| CD3DN, % CD3 | 27.27 | 27.59 | 37.04 | 48.82^{e} | 28.67 | Shi et al. |
| NK cells, % MNC | 8.8 | 13.43 | 12.81 | 22.65^{e} | 5.60^{e} | Beldi-Ferchiou |
| | | | | | | and Caillat- |
| | | | | | | Zucman |
| NKT cells, % MNC | 37.68 | 41.91 | 37.1 | 45.89^{e} | 34.78 | Chen et al. |
| NKT-CD8, % NKT cells | 34.08 | 37.29 | 30.1 | 40.49 | 29.38 | Chen et al. |
| B cells, % MNC | 1.16 | 2.58^{e} | 1.1 | 22.87^{e} | 1.38 | Fife and Bluestone |
| LIN neg, % MNC | 0.21 | 0.35 | 0.2 | . . . | 0.2 | Liu et al. |
| Monocytes, % MNC | 0.19 | 0.07^{e} | 0.21 | 4.27^{e} | 0.13 | Chen |
| Granulocytes, % CD45 pos | 0.87 | 0.92 | 1 | 3.94^{e} | 0.57^{e} | Orozco-Uribe et al. |

LIN neg, lineage negative; pos, positive.
^{a}NKT-DP, NKT-CD4, and NKT-DN populations in all cohorts and LIN neg for the CLL cohort were not included in the analysis because they did not reach the minimum criteria for event counts.
^{b}Bold indicates populations determined positive (≥5%) for PD-1 or CTLA-4.
^{c}Contains examples of phenotypes previously observed.
^{d}Dash indicates that examples were not found.
^{e}Value significantly higher or lower than HV (P < 0.05).

### REFERENCES

Anand et al., Lupus 2002; 11: 493-500
Beldi-Ferchiou and Caillat-Zucman, Int J Mol Sci 2017; 18
Chauhan et al., J Med Virol 2012; 84: 845-856
Chen, Nat Rev Immunol 2004; 4: 336-347
Chen et al., Oncoimmunol 2016; 5: e1176653
Fife and Bluestone, Immunol Rev 2008; 224: 166-182
Foldi et al., J Infect Dis 2017; 216: 641-650
Fujiwara et al., PLoS Negl Trop Dis 2009; 3: e399
Jago et al., Clin Exp Immunol 2004; 136: 463-471
Kaufmann et al., Nature Immunol 2007; 8: 1246
Liu et al., Cancer Immunol Immunother 2009; 58: 687-697
Long et al., J Clin Invest 2017; 127: 3052-3064
Orozco-Uribe et al., Tuberculosis-Expanding Knowledge: InTech; 2015
Pardoll, Nat Rev Cancer 2012; 12: 252-264
Shi et al., J Hematol Oncol 2013; 6: 74
Steiner et al., Clin Exp Immunol 2001; 126: 143-150
Waki et al., Cancer Sci 2014; 105: 1229-1235
Wang et al., Scand J Immunol 2002; 55: 53-60
Yamagiwa et al., Hepatol Res: Official J Japan Soc Hepatol 2008; 38: 664-672
Yang et al., Blood Cancer J2015; 5: e281

TABLE 2
Patient Characteristics

n (range)

| Characteristics | Pembrolizumab | Atezolizumab |
|---|---|---|
| Median age (years) | 70 (50-84) | 70 (46-77) |

Gender

| | | |
|---|---|---|
| Male | 8 | 4 |
| Female | 8 | 6 |

Cancer

| | | |
|---|---|---|
| Lung | 11 | 10 |
| Genitourinary | 1 | |
| Melanoma | 4 | |

Response

| | | |
|---|---|---|
| Benefit | 7 | 4 |
| Complete response | 3 | 1 |
| Progressive disease | 6 | 5 |

TABLE 3
Phenotypic Descriptions of PD-1 and CTLA-4 expressing cells

| Mononuclear cells | SSCmed&lo CD45+ |
|---|---|
| T cells | CD45+CD3+ |
| CD4+ T cells | CD45+CD3+CD4+ |
| Naïve CD4+ | CD45+CD3+CD4+CD45RO− |
| Memory CD4+ | CD45+CD3+CD4+CD45RO+ |
| CD8+ T cells | CD45+CD3+CD8+ |
| Naïve CD8+ | CD45+CD3+CD8+CD45RO− |
| Memory CD8+ | CD45+CD3+CD8+CD45RO+ |
| Double Positive (DP) T cells | CD45+CD3+CD8+CD4+ |
| Double Negative (DN) T cells | CD45+CD3+CD8−CD4− |
| NK cells | CD45+CD56+ |
| NKT cells | CD45+CD3+CD56+ |
| NKT CD8+ | CD45+CD3+CD56+CD8+ |
| NKT DP | CD45+CD3+CD56+CD8+CD4+ |
| NKT CD4+ | CD45+CD3+CD56+CD4+ |
| NKT DN | CD45+CD3+CD56+CD8−CD4− |
| B cells | SSClo CD45med (CD3−CD4−CD56−) |
| Lin neg cells | SSClo CD45lo (CD3−CD4−CD56−) |
| Monocytes | SSCmed CD45med (CD3−CD4−CD56−) |
| Granulocytes | SSChi CD45+ |

### Example 6—PD-1 Based Clustering and Profiling

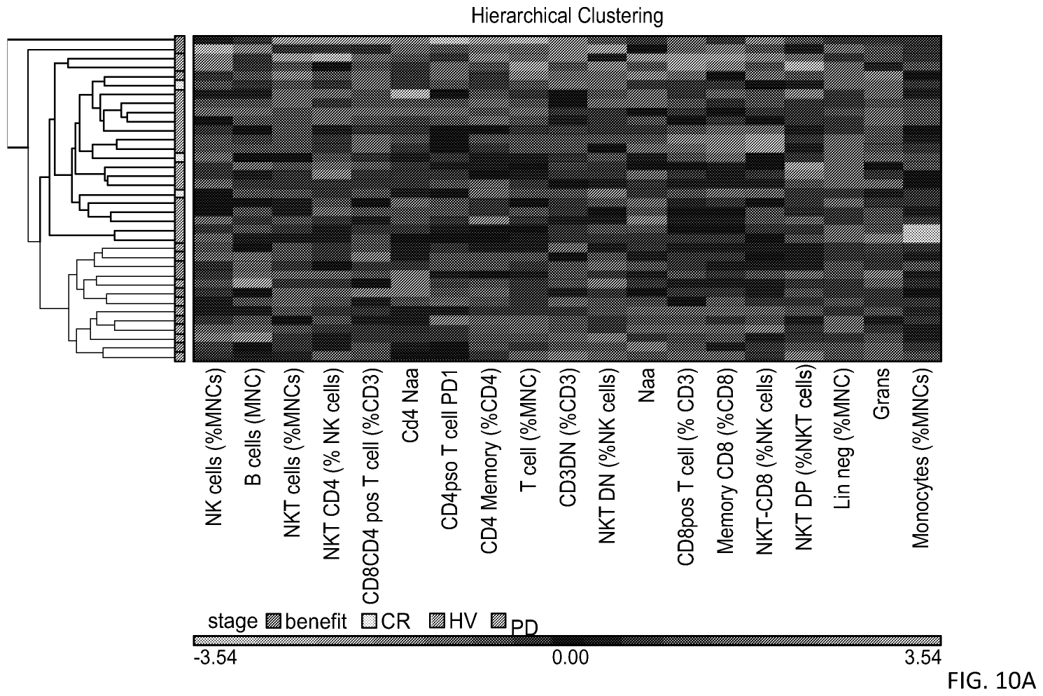

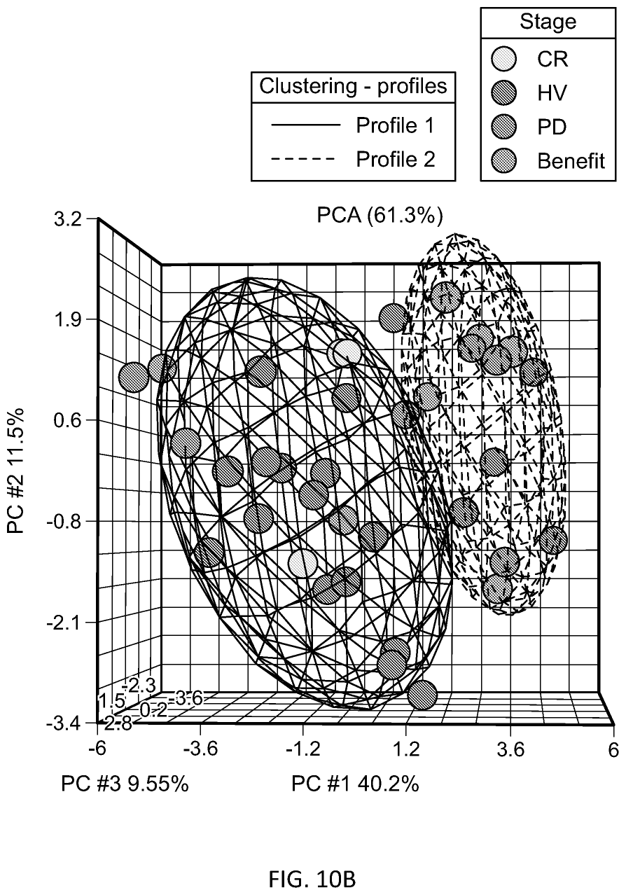

A flow cytometry-based assay was used to measure PD-1 levels on 19 peripheral blood leukocyte populations of cancer patients treated with pembrolizumab and a group of HV using. Hierarchical clustering was performed on pre-treatment values of 16 cancer patients (11 lung, 1 GU, 4 melanoma) and 20 healthy volunteers. Two unique profiles were identified (FIG. 10A and TABLE 4). Profile 1 consisted of three CR, two PD, and 16 HVs, while profile 2 had five patients that benefitted from treatment, four with PD, and four HVs. Two patients who benefitted from treatment did not fit into either profile. Principle component analysis was used to determine which phenotypes were the greatest contributors to each profile, with the most influential being PD-1 levels on T cells (PC1), Granulocytes (PC2) and CD4 Naïve T cells (PC3) (FIG. 10B and TABLE 5). This analysis demonstrated that patients who cluster into profile 1 have a greater chance for complete response to pembrolizumab.

TABLE 4
PD-1 profiles

| Profile | (n) | CR | Benefit | PD | HV |
|---|---|---|---|---|---|
| Profile 1 | 21 | 3 | 0 | 2 | 16 |
| Profile 2 | 13 | 0 | 5 | 4 | 4 |
| Other | 2 | 0 | 2 | 0 | 0 |

TABLE 5

| PC1 | | PC2 | | PC3 | |
|---|---|---|---|---|---|
| T cell | 0.8854 | Grans | 0.7335 | CD4 Naïve | 0.4882 |
| (% MNC) | | | | (% CD4) | |
| NKT cells | 0.8595 | Monocytes | 0.6935 | CD4pos T | 0.4695 |
| (% MNC) | | (% MNC) | | cell PD1 | |
| CD8pos T | 0.7790 | Lin neg | 0.6342 | CD4 | 0.3728 |
| cell | | (% MNC) | | Memory | |
| (% CD3) | | | | (% CD4) | |
| CD8CD4pos | 0.7769 | NKT DP | 0.2618 | NKT DN | 0.3096 |
| T cell | | (% NKT | | (% NK | |
| (% CD3) | | cells) | | cells) | |
| CD4 Memory | 0.7594 | CD4 Naïve | 0.2259 | NK cells | 0.2132 |
| (% CD4) | | (% CD4) | | (% MNC) | |

### Example 7—Single Analyte Immune Data Score (SAID)

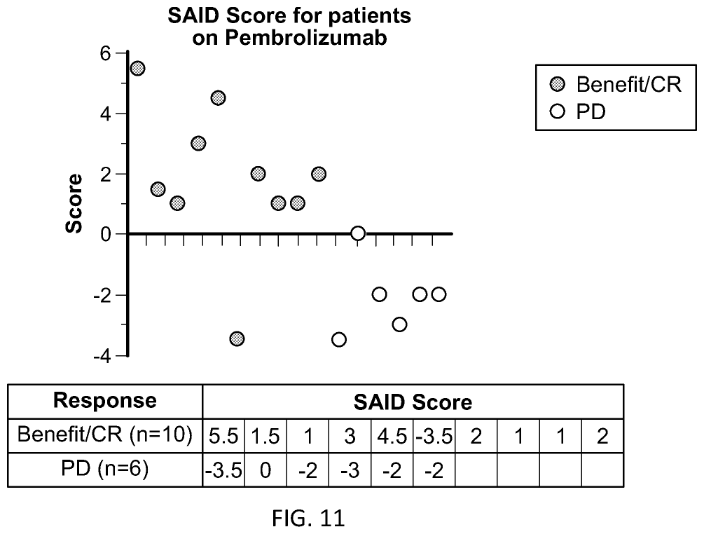

A scoring system was developed based on the single analyte immune phenotyping data. Of the 13 phenotypes that were found to have significant differences between HVs, patients that had a complete response or benefitted from pembrolizumab, and those who had progressive disease, 11 were weighted and used to determine the SAID score. Phenotypes were weighed most heavily if they showed significant differences between benefit/CR and PD as they were determined to be a major biomarker for prognosis. Next, the phenotypes were ranked based on p values. Those with 0.001 significance were weighed twice as heavily as those with 0.05. Cut off values for each analyte were determined based on 95% CI. Values indicative of PD were assigned a negative score while those indicating positive response were assigned a positive score. The negative and positive values, when combined, totaled 1 (TABLE 6). A patient with a score of 1 or greater has a 93% chance of benefitting from pembrolizumab. This scoring system was tested on 16 patients treated with pembrolizumab. Of the patients that benefitted or had a full recovery, 9 out of 10 had a score of 1 or greater (FIG. 11). For patients that had progressive disease, 6 out of 6 had scores of zero or lower (FIG. 11).

TABLE 6

| Single Analyte Phenotype | Value | Score |
|---|---|---|
| PD1+CD3+DN (% CD3) | 19 or above | −2.5 |
| PD1+CD8+Naïve (% CD8) | 10 or above | +1 |
| Grans (cells/μL) | 4500 or above | +1 |
| Neutrophils 15+16+ (cells/μL) | 4300 or above | +1 |
| Grans (% CD45) | 78 or above | +1 |
| Eosinophils 15+16− (cells/μL) | 271 or above | +0.5 |
| PD1+CD3+ (% MNC) | 13 or above | +0.5 |
| PD1+CD8+ (% CD3) | 26 or above | +0.5 |
| Intermediate Monocytes 14+16+ (cells/μL) | 88 or above | −1 |
| CD3 (% MNC) | 52 or above | −0.5 |
| Monocytes (% CD45pos) | 35 or above | −0.5 |

| | |
|---|---|
| Total | 1 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-24. (canceled)

25. A method for treating a mammal having cancer, wherein said method comprises:
(a) identifying said mammal as having an elevated level of an immune phenotype associated with a likely response to treatment with pembrolizumab, as compared to a control level of said immune phenotype, and
(b) administering, to said mammal, or instructing said mammal to self-administer, a composition comprising pembrolizumab.

26. The method of claim 25, wherein said mammal is a human.

27. The method of claim 25, wherein said immune phenotype is selected from the group consisting of PD1+CD8+ Naïve (% CD8), number of granulocytes (Grans) (cells/μL), number of Neutrophils 15+16+ (cells/μL), Grans (% CD45), Eosinophils 15+16− (cells/μL), PD1+CD3+ (% MNCs), and PD1+CD8+ (% CD3).

28. The method of claim 25, wherein said cancer is selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

29. A method for treating a mammal having cancer, wherein said method comprises administering a composition comprising pembrolizumab to a mammal identified as having the an elevated level of an immune phenotype associated with a likely response to pembrolizumab treatment, as compared to a control level of said immune phenotype.

30. The method of claim 29, wherein said mammal is a human.

31. The method of claim 29, wherein said immune phenotype is selected from the group consisting of PD1+CD8+ Naïve (% CD8), number of granulocytes (Grans) (cells/μL), number of Neutrophils 15+16+ (cells/μL), Grans (% CD45), Eosinophils 15+16− (cells/μL), PD1+CD3+ (% MNCs), and PD1+CD8+ (% CD3).

32. The method of claim 29, wherein said cancer is selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

33. A method for treating a mammal having cancer, wherein said method comprises:
(a) identifying said mammal as having an elevated level of an immune phenotype associated with a lack of response to treatment with pembrolizumab, as compared to a control level of said immune phenotype, and
(b) administering, to said mammal, or instructing said mammal to self-administer, a composition comprising a therapeutic agent other than pembrolizumab.

34. The method of claim 33, wherein said mammal is a human.

35. The method of claim 33, wherein said immune phenotype is selected from the group consisting of PD1+CD3+ DN (% CD3), Intermediate Monocytes 14+16+ (cells/μL), CD3 (% MNCs), and Monocytes (% CD45pos).

36. The method of claim 33, wherein said cancer is selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

37. A method for treating a mammal having cancer, wherein said method comprises administering a composition comprising a therapeutic agent other than pembrolizumab to a mammal identified as having an elevated level of an immune phenotype associated with a lack of response to pembrolizumab treatment, as compared to a control level of said immune phenotype.

38. The method of claim 37, wherein said mammal is a human.

39. The method of claim 37, wherein said immune phenotype is selected from the group consisting of PD1+CD3+ DN (% CD3), Intermediate Monocytes 14+16+ (cells/μL), CD3 (% MNCs), and Monocytes (% CD45pos).

40. The method of claim 37, wherein said cancer is selected from the group consisting of liver cancer, lung cancer, kidney cancer, brain cancer, genitourinary cancer, ovarian cancer, prostate cancer, bladder cancer, thyroid cancer, leukemia, lymphoma, head and neck cancer, bone cancer, stomach cancer, breast cancer, sarcomas, and melanoma.

41. A method for determining a Single Analyte Immune Data (SAID) score for a mammal with cancer, said method comprising:
measuring, in a biological sample from the mammal, the following immune phenotypes:
PD1+CD3+ DN (% CD3);
PD1+CD8+ Naïve (% CD8);
Granulocytes (Grans) (cells/μL);
Neutrophils 15+16+ (cells/μL);
Grans (% CD45);
Eosinophils 15+16− (cells/μL);
PD1+CD3+ (% MNC);
PD1+CD8+ (% CD3);
Intermediate Monocytes 14+16+ (cells/μL);
CD3 (% MNC); and
Monocytes (% CD45pos),
assigning a first score of −2.5 when said PD1+CD3+ DN (% CD3) is 19 or above, or assigning a first score of 0 when said PD1+CD3+ DN (% CD3) is less than 19;
assigning a second score of +1 when said PD1+CD8+ Naïve (% CD8) is 10 or above, or assigning a second score of 0 when said PD1+CD8+ Naïve (% CD8) is less than 10;
assigning a third score of +1 when said Grans (cells/μL) is 4500 cells/μL or above, or assigning a third score of 0 when said Grans (cells/μL) is less than 4500;
assigning a fourth score of +1 when said Neutrophils 15+16+ (cells/μL) is 4300 or above, or assigning a fourth score of 0 when said Neutrophils 15+16+ (cells/μL) is less than 4300;
assigning a fifth score of +1 when said Grans (% CD45) is 78 or above, or assigning a fifth score of 0 when said Grans (% CD45) is less than 78;
assigning a sixth score of +0.5 when said Eosinophils 15+16− (cells/μL) is 271 or above, or assigning a sixth score of 0 when said Eosinophils 15+16− (cells/μL) is less than 271;
assigning a seventh score of +0.5 when said PD1+CD3+ (% MNC) is 13 or above, or assigning a seventh score of 0 when said PD1+CD3+ (% MNC) is less than 13;
assigning an eight score of +0.5 when said PD1+CD8+ (% CD3) is 26 or above, or assigning an eighth score of 0 when said PD1+CD8+ (% CD3) is less than 26;
assigning a ninth score of −1 when said Intermediate Monocytes 14+16+ (cells/μL) is 88 or above, or assigning a ninth score of 0 when said Intermediate Monocytes 14+16+ (cells/μL) is less than 88;
assigning a tenth score of −0.5 when said CD3 (% MNC) is 52 or above, or assigning a tenth score of 0 when said CD3 (% MNC) is less than 52;
assigning an eleventh score of −0.5 when said Monocytes (% CD45pos) is 35 or above, or assigning an eleventh score of 0 when said Monocytes (% CD45pos) is less than 35; and
calculating said SAID score by totaling said first score through said eleventh score.

42-44. (canceled)
